(19) United States
(12) Patent Application Publication
(10) Pub. No.: US 2007/0213693 A1
(43) Pub. Date: Sep. 13, 2007

(54) SELECTIVE OPHTHALMIC LASER TREATMENT

(75) Inventor: Malcolm Plunkett, Crafers (AU)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(73) Assignee: ELLEX MEDICAL PTY LTD, Adelaide, SA (AU)

(21) Appl. No.: 11/574,270

(22) PCT Filed: Aug. 24, 2005
(86) PCT No.: PCT/AU05/01273

§ 371 Date: Feb. 26, 2007

(30) Foreign Application Priority Data
Aug. 27, 2004 (AU) 2004904884

(51) Int. Cl. A61F 9/008; A61B 18/18

(52) U.S. Cl. 606/6; 606/4

(57) ABSTRACT

An ophthalmic laser system which produces controlled bursts of laser pulses and incorporates a system control processor that calculates the likely tissue effects and the total treatment time based on selected laser treatment parameters. The system incorporates a graphical user interface that displays the likely tissue effects to the user (ophthalmic surgeon) to assist with selection of optimal treatment parameters. The system and method of operation is particularly useful for procedures such as selective retinal therapy by displaying a therapeutic window in which treatment of target tissue is achieved without damage to surrounding tissue.

This invention relates to a method of ophthalmic treatment and a laser instrument designed for use by ophthalmologists for performing the treatment. In particular, the invention relates to a laser system and treatment method for selective ophthalmic laser treatment of ocular structures and individual retinal layers, such as the retinal pigmented epithelium (RPE).

### BACKGROUND TO THE INVENTION

Ophthalmic laser systems are being used for an ever increasing variety of procedures for treating various eye disorders. Equipment and methods for treating glaucoma and performing secondary cataract surgery have been described in our co-pending international application WO 04/027487 titled “Ophthalmic Laser System”. We have also described a laser system designed for Retinal Photocoagulation, Pan Retinal Photocoagulation, Photocoagulation for Macular Degeneration and Laser Trabeculoplasty in published international application number WO 02/083041.

Although these laser systems have proven to be extremely useful for their intended purpose, it has been realised that a number of ophthalmic procedures may cause unintended collateral damage to parts of the eye. For instance, many treatments rely upon heating to cause photocoagulation in a target area. Unfortunately these methods generally rely upon the appearance of a visible lesion on the surface of the eye as an indication that the desired photocoagulation has occurred within the target area. Because a large proportion of the laser radiation is absorbed in layers below the surface of the retina, considerable damage is done to sub-surface retinal layers before a visible lesion appears. While many retinal diseases can be treated by this method the benefit obtained must be carefully considered in relation to the lesions and sub-surface damage that occurs, which can result in a severe loss of visual acuity.

Several retinal diseases are thought to be initiated by a reduction in the correct functioning of the RPE and studies have shown that the function of the retina can be improved by damaging the sub-surface mono-layer of cells in the RPE layer and then allowing them to rejuvenate, but only if it can be done without damaging the overlying neuro-retinal layers or the underlying choroid. The photoreceptors in the neuro-retina are particularly sensitive to damage, however 50% of the incident light that falls on the retina is absorbed in the RPE layer due to their high melanosome content. This makes it possible to selectively heat the RPE layer by applying suitable laser energy, however it is difficult to confine the temperature rise within the RPE to a level that can damage the RPE cells without damaging the photoreceptors.

Attempts have been made (Clinical Applications of the MicroPulse Diode Laser, Moorman C M, Hamilton A M P, Eye 13:145-150, 1999) to selectively treat the RPE layer using a number of 810 nm laser pulses which are about 100 μs in duration at energy levels which did not cause visible lesions. While the objective of this was to spatially confine the temperature rise within the RPE layer, the duration of the pulses is thought to be too long to prevent damage to the neuro-retinal structures immediately adjacent to the RPE layer.

To attempt to overcome this limitation a number of methods have been developed to monitor the effect of the laser treatment at the target area in an effort to provide the ophthalmologist with an accurate treatment end-point which causes the least collateral damage. One such method is described in U.S. Pat. No. 6,540,391 assigned to Iridex Corporation, which describes an interferometric technique to monitor the changes in the target area during the course of a treatment. The method is difficult to implement and limited in application.

The same company describes an intra-operative monitoring system that measures focal electroretinograms during the course of laser treatment to provide a feedback measure to the physician. The approach is described in U.S. Pat. No. 6,733,490. As with the interferometric technique, the measurement and analysis of focal electroretinograms is difficult to implement and of limited application.

A third approach described by Iridex Corporation is described in international patent publication number WO 04/026099. In this patent application they propose a technique in which light scattering is monitored during a treatment on the basis that changes in scattering intensity correlate with temperature dependent changes at the treatment site.

Each of the techniques described by Iridex Corporation add cost and complexity and do not address the fundamental problem of thermal damage to the photoreceptors caused by the relatively long laser pulse duration used.

An alternate approach is presented by Roider, Brinkmann, Wirbelauer, Laqua and Birngruber in J. Ophthalmol. 2000 84:40-47 in which a series of 527 nm laser pulses of 1.7 μs duration are used to selectively treat the RPE layer while avoiding unwanted collateral effects. The use of this pulse duration allows rapid temperature rise in the melanosites within the RPE cells but limits heat diffusion to the photoreceptors. This approach is based on the work of Birngruber in U.S. Pat. No. 5,302,259 which describes a method of coagulating material based on selective absorption of energy. Although Birngruber describes the general principle in his patent, he does not provide description of how to put this into practical effect for ophthalmic laser treatment but does show in his paper that selective ophthalmic laser treatment applications are possible by separately evaluating each patient and then carefully balancing a variety of laser parameters to achieve the optimal treatment.

It is evident that the concepts described by Birngruber must be refined to achieve a practical device that can be routinely used for ophthalmic laser treatments.

An attempt at such a refinement is found in U.S. Pat. No. 5,549,596 in the name of Latina. Latina applies the Birngruber technique to selectively target pigmented ocular cells to treat glaucoma, intraocular melanoma and disease of the RPE. The claims of the Latina patent describe the use of a radiant exposure of 0.01 J/cm² to 5 J/cm² using one or more pulses which are 1 ns to 2 μs in duration and using a wavelength that is absorbed more in the pigmented cells than in the non-pigmented cells.

The Latina technique has been successfully applied in the treatment of glaucoma using a procedure known as selective laser trabeculoplasy (SLT). SLT treatment is applied to areas of melanin concentration in the trabecular meshwork (TM) in order to reduce the intraocular pressure. The TM can be directly accessed by the laser radiation without the need to pass through any overlying tissue so in this case the radiant exposure range of 0.01 J/cm² to 5 J/cm² is adequate, however clinical results have shown that this radiant exposure range is insufficient to effectively treat the RPE layer and that the combination of other laser pulse parameters such as the number of pulses and the pulse repetition rate are critical in achieving effective coagulation of the RPE layer while sparing the neuro-retina and choroid.

It appears from the published work on selective laser trabeculoplasy and sub-threshold retinal laser treatment that careful control of laser energy delivery can facilitate a new level of ophthalmic laser treatment precision and sophistication. While some laser systems allow partially selective RPE treatment, and the effectiveness of highly selective treatment have been experimentally demonstrated in a limited manner, there are no laser systems or treatment protocols that can produce the full range of treatment options required in a manner that can be readily understood by the ophthalmologist, so that the full potential of selective ophthalmic laser treatment can be realized.

### DISCLOSURE OF THE INVENTION

In one form, although it need not be the only or indeed the broadest form, the invention resides in an ophthalmic laser system comprising:
a laser module producing laser pulses with a pulse repetition rate capable of causing an additive thermal effect within target tissue, a pulse duration capable of containing thermal diffusion substantially within the target tissue, and a wavelength chosen to optimize energy delivery to the target tissue;
a control module in signal connection with the laser module and incorporating means for controlling said laser module to deliver a selected number of pulse bursts of selected duration and selected repetition rate with controlled pulse energy so that pulses within each burst have an additive thermal effect within the target tissue to cause an incremental temperature rise while limiting thermal diffusion to adjacent structures; and
a delivery module in optical connection with said laser module and signal connection with the control module, said delivery module delivering said bursts of laser pulses with a controlled radiant energy to a treatment zone.

The laser module of the ophthalmic laser system suitably incorporates a pulsed laser and a pulse gating element, said pulsed laser producing a train of pulses and said pulse gating element selecting bursts of pulses from said train of pulses.

The pulsed laser is suitably a Q-switched solid state laser operating in the wavelength range from 500 nm to 750 nm with a pulse repetition rate from 1 kHz to 500 kHz, and a pulse duration from 0.1 μs to 40 μs.

The ophthalmic laser system may further comprise feedback means that provides treatment feedback to the control module for dynamic control of the laser module.

In a further form the invention resides in a method of ophthalmic laser treatment including the steps of:
selecting laser treatment parameters;
automatically calculating and displaying a likely selectivity of a treatment which will result from the laser treatment parameters;
automatically calculating and displaying a total treatment time based on the laser treatment parameters;
adjusting said laser treatment parameters to achieve a desired selectivity and total treatment time; and
controlling a laser system according to said laser treatment parameters to deliver laser pulses to a treatment zone.

The method is preferably applied to the retinal pigmented epithelium layer in a procedure such as Selective Retinal Therapy (SRT).

The method may further include the step of selecting treatment target values, if these have been pre-determined, and displaying the target treatment values with the calculated sensitivity and treatment time. The treatment target values may be derived from patient dependant pre-set variables and measured values.

The invention may further include using a visible lesion threshold to determine estimated optimal laser treatment parameters by:
selecting laser treatment parameters intended to cause a visible lesion at a periphery of a retina;
selecting patient dependant pre-set variables including a Visual Lesion Threshold scaling factor;
controlling and activating a laser system to deliver a selected series of laser pulses to the periphery of the retina;
adjusting the laser treatment parameters to determine the Visible Lesion Threshold; and
calculating and displaying the estimated optimal laser treatment parameters and tissue temperature rise targets for selective treatment based on the Visible Lesion Threshold and Visible Lesion Threshold scaling factor.

Further manual adjustment of treatment parameters by a user may be required. Once the estimated optimal laser treatment parameters are determined the method proceeds as above.

The invention may further include using feedback from external measurement devices, which are designed to indicate the effectiveness of ophthalmic laser treatment, to allow manual or automatic adjustment of laser treatment parameters to optimize the treatment by:
connection of a laser system to an external measurement device which can provide feedback on the effectiveness of selective treatment; and
displaying treatment effectiveness based on the external measurement device and automatic or manual adjustment of treatment parameters to optimize the selective treatment.

### BRIEF DESCRIPTION OF THE DRAWINGS

To assist in understanding the invention, preferred embodiments will be described with reference to the following figures in which:

FIG. 1 shows a general block diagram of an ophthalmic laser system for photocoagulation;

FIG. 2 shows a detailed view of the laser module of FIG. 1;

FIG. 3 shows a detailed view of the delivery module of FIG. 1;

FIG. 4 shows a detailed view of the control module of FIG. 1;

FIG. 5 is a schematic cross section of a human retina showing treatment zones;

FIG. 6 is a graphical user interface for treatment aid software; and

FIG. 7 is a simplified flowchart of the thermal modeling algorithms used in the graphical user interface.

### DETAILED DESCRIPTION OF THE DRAWINGS

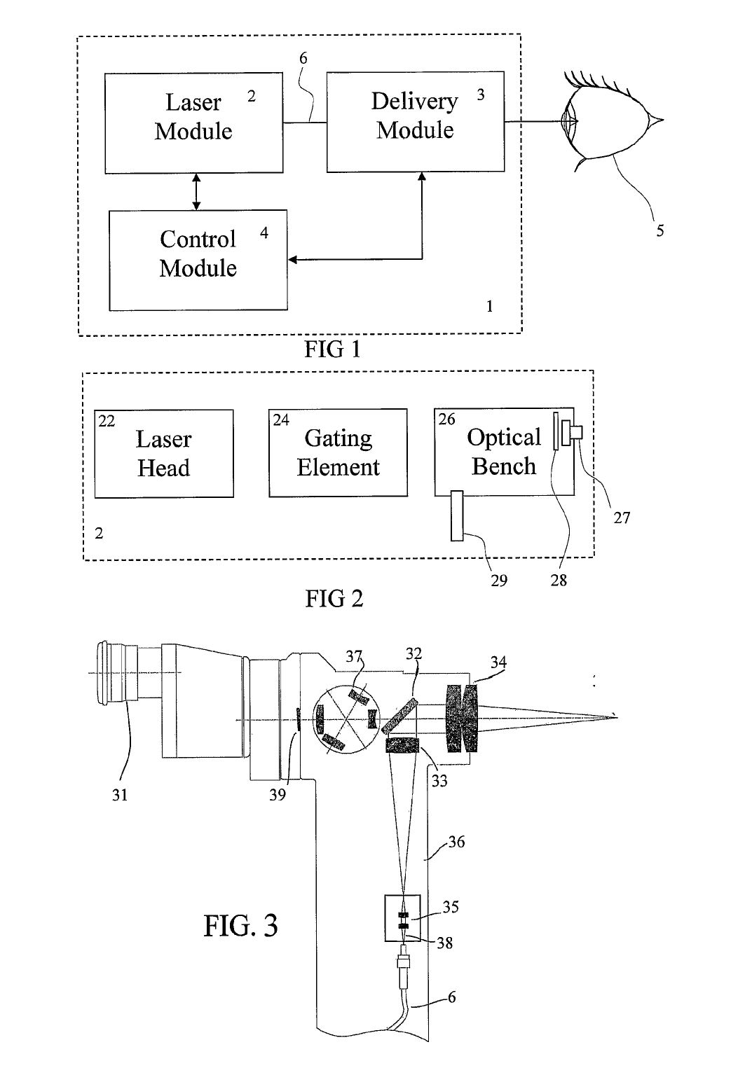

Referring to FIG. 1, there is shown an embodiment of an ophthalmic laser system 1 useful for a selectable range of photocoagulation procedures, such as Selective Retinal Therapy (SRT), Selective Laser Trabeculoplasty (SLT), Iridotomy and non-selective retinal coagulation. The system is comprised of three main modules being a laser module 2, delivery module 3 and control module 4. The laser module 2 delivers a controlled burst of laser pulses of known energy, wavelength, duration and repetition rate. The output of the laser module is delivered to a treatment area 5 via the delivery module 3. The control module 4 provides power to the laser module 2 and control signals to and from both the laser module 2 and the delivery module 3 to control the parameters of laser radiation delivered to the treatment zone 5. A fiber optic 6 guides the output from the laser module 2 to the delivery module 3.

A preferred embodiment of the laser module 2 is shown schematically in FIG. 2. The module comprises the laser head 22, a pulse gating element 24 and an optics bench 26. The laser head 22 is suitably a Q-switched solid state laser generating a continuous train of short pulses which are selected in bursts by the pulse gating element 24. Other laser systems with similar operating parameters will also be suitable. For instance, the inventors enviage that the pulse gating element could be embodied as a fast switching of power supply of a laser diode pump source for a solid state pulsed laser module.

The laser operates between 500 nm and 750 nm. The appropriate solid state active medium is selected for the required wavelength. An active medium of Nd:YAG can produce 532 nm, 561 nm or 659 nm and Nd:YLF can produce 527 nm. A typical laser for the invention is a frequency doubled Nd:YAG laser operating with the following parameters:
Wavelength: 532 nm
Pulse duration: 1 μsec (fixed)
Energy per pulse: 0.5 μJ to 70 μJ (adjustable)
Pulse Repetition Rate: 30 kHz (Q-switch rate)

The pulse gating element allows the required combination of pulses in a burst to be delivered to the treatment zone via the optics bench module and the delivery module. The required combination of pulses is determined by the system control module in response to the user settings. The pulse gating element is typically an electro-optic switch. Typical operating parameters for use with the laser head described above are:
Pulse burst repetition rate: 0.1 kHz to 5 kHz (adjustable)
Pulses per burst: 1 to 500 (adjustable)

For example, when the intra-cavity Q-switch frequency is 30 kHz (rep rate of 33.3 μs per pulse ) and the total treatment time is 100 ms, the laser will output about 3000 pulses. The pulse gating element can then be operated to pass any combination of pulses in a burst. It could, for example, be controlled to pass 3 pulses every 1 ms thus giving about 100 bursts with 3 pulses per burst.

The optical bench 26 has optics for coupling the output from the laser 22 and gating element 24 to the optical fiber 6 via optical fiber coupler 27. It also includes a safety shutter 28 that blocks the optical fiber coupler under control of the control module 4. An aiming laser 29 may be provided on the optical bench 26 and aligned to be coaxial with the output of the laser 22.

A suitable delivery module 3 is shown in FIG. 3. The delivery module 3 incorporates a binocular viewing microscope 31 and alignment optics including folding mirror 32, micromanipulator lens 33, objective lens 34, safety filter 39 and optical zoom 35. A magnification changer 37 is optionally included. The delivery module is suitably incorporated in a microscope support arm 36. The optical fiber 6 is substantially, and preferably entirely, enclosed within the microscope support arm. These elements have been described previously in our application WO 03/083041, mentioned above.

The micromanipulator lens is mounted on a pivotable arm, wherein pivoting of said lens about an optic axis translates to movement of a focused output of the optical fibre at the treatment zone 5.

The position of the optical zoom 35 can be adjusted by the user to set the spot size at treatment zone 5 and the zoom position is monitored by the control module 4 for use in setting the laser parameters to deliver a desired total radiant energy. The spot size determines the total radiant energy that is delivered at the treatment zone. In some embodiments the optical zoom 35 may also be automated and set directly by the control module 4. Persons skilled in the field will be aware of various linear drive and stepper motor options that are useful for automating the optical zoom.

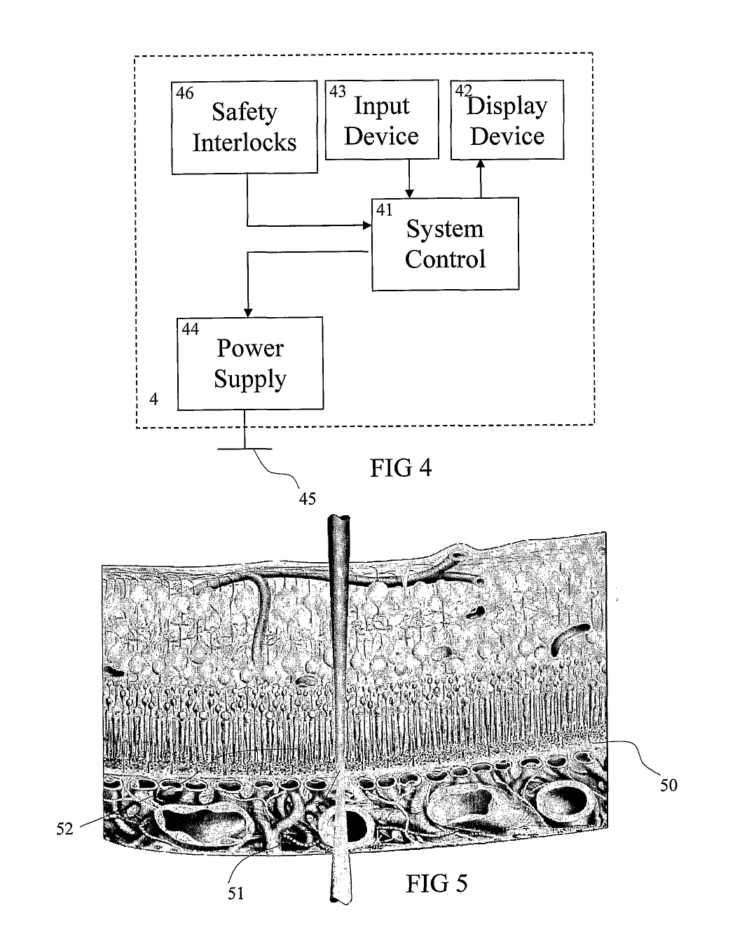

The control module is shown in greater detail in FIG. 4. The control module 4 allows the user to select from a range of laser operating modes to suit a particular treatment. A system control processor 41 runs algorithms to calculate likely tissue effects and treatment time and to control operation of the ophthalmic laser system. The algorithms are described in greater detail below. A display 42 indicates the current operating parameters to the user. An input device 43, such as a keypad, allows the user to select from a range of pre-set treatments or to input custom parameters. The various modes of operation are discussed below in greater detail.

The control module 4 also incorporates a power supply 44 that converts mains power 45 to all voltages required in the control module 4, delivery module 3 and the laser module 2. Various interlocks 46 ensure safe operation of the system.

In use, the user can select various treatment modes via the input 43 including, selective RPE treatment, selective trabecular meshwork treatment, Iridotomy, and non-selective retinal coagulation. The system control processor 41 displays the selected treatment parameters and likely treatment outcome in a manner that suits the selected treatment mode and then, on command of the user, delivers the selected treatment as a series of laser pulses that are controlled by the intra-cavity Q-switch within the laser module and the pulse gating element.

The selective RPE treatment mode is the most demanding mode as the target RPE layer is a sub-surface layer. Spatial confinement of the temperature rise in the RPE layer is required to produce selective photo-coagulation, which requires careful control of the energy delivery. The laser pulse duration must be well below the thermal relaxation time of the target structure to avoid heat diffusion into adjacent structures which could cause collateral damage. This results in the need to deliver the high energy levels that can produce localized photo-coagulation in a very short time period.

To avoid inducing mechanical effects such as cavitations and micro-explosions due to the resulting high energy gradients it is preferable to deliver the energy as a series of very short duration bursts of laser pulses, with a relatively low repetition rate, which have an additive thermal effect. For example, selective RPE treatment may require up to 300 μJ pulses with 1 μs duration, which are repeated every 2 ms. Rather than deliver these as single high energy pulses, the laser system presented is able to deliver bursts of closely spaced laser pulses which can have the same additive effect as a single high energy pulse. The delivery of pulse bursts reduces the cost and complexity of the laser system and further reduces the risk of unwanted mechanical effects at the treatment zone. The intra-cavity Q-switch within the laser module produces pulses at the pulse burst rate, while the pulse gating module allows the burst repetition rate and the number of pulses per burst to be controlled.

The radiation is delivered to the retina and other ocular structures in bursts of laser pulses of a wavelength between about 500 nm and about 750 nm, which is preferentially absorbed more in the target layer or ocular structure than in adjacent areas for selective treatment modes, with pulse durations of between 0.1 μs and 40 μs, energy per pulse up to approximately 300 μJ , pulse repetition rate of between 1 kHz and 500 kHz, pulse burst repetition rate of between 0.05 kHz, and 5 kHz, pulses per burst of between 1 and 100 pulses, and between 1 and 500 pulse bursts. By selecting the number of laser pulse bursts, the burst repetition rate, the number of pulses per burst, the laser pulse intensity and treatment area to achieve a total radiant exposure of between about 1 and about 300 Joules/cm², it is possible to heat the target layers or ocular structures within the selected treatment area to a temperature that causes damage to it without causing a temperature rise that can damage the adjacent layers or ocular structures. Alternatively, other combinations of pulse bursts, pulse burst intervals and pulse energy levels can be chosen to produce other selective or non-selective photo-coagulation effects to suit other treatment modes.

By choosing a treatment radiation wavelength which is close to the lower end of the stated range of 500 nm to 750 nm, the minimum treatment energy can be used because of maximum absorption within the melanin of the RPE layer, however the wavelength of the treatment radiation can also be chosen to minimize the interference from overlying retinal vasculature. A wavelength which is close to the higher end of the stated range, such as 670 nm, can be used which has minimum absorption in oxygenated hemoglobin, which will result in more consistent energy delivery to the treatment spot area of the RPE layer and reduce the chance of retinal vascular damage.

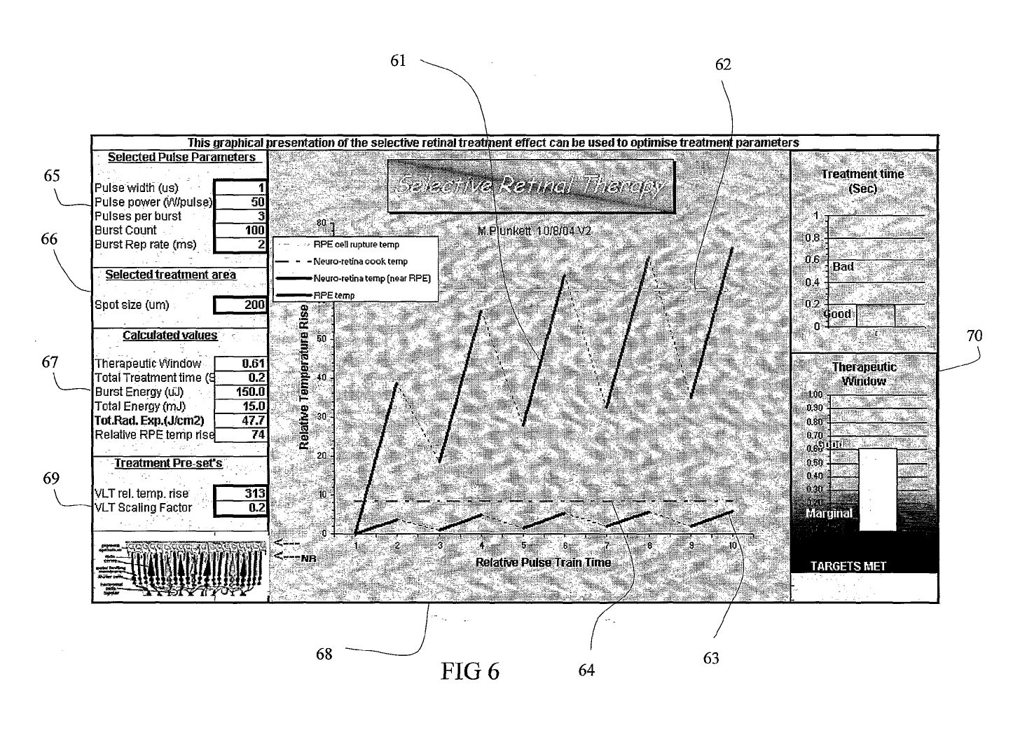

In a preferred embodiment the laser is employed for a method of treating the retinal pigmented epithelium (RPE) layer. To obtain selective photocoagulation of the RPE layer a large amount of energy must be delivered in a short time, and then repeated a number of times with a relatively large time between pulses. Typical values are a wavelength of 532 nm, 1 μs pulse duration, 3 pulses per burst, 30 kHz pulse repetition rate, 50 μJ pulse energy, 500 Hz pulse burst repetition rate and a total of 100 bursts. Using a 200 micron diameter treatment spot this will produce a total radiant exposure of about 48J/cm², as shown in FIG. 6. At any time the user can select treatment parameters such as pulse burst energy, pulse burst repetition rate, number of bursts per treatment and the spot size. When the user selects the selective RPE treatment mode, the parameters chosen by the user are analysed by the system control processor 41 and the calculated likely therapeutic window, total treatment time and likely temperature rise characteristics for the RPE layer and adjacent Neuro-retina are calculated and displayed. Any changes to the treatment parameters by the user will cause the display of the calculated values to be updated. The user can then use the calculated likely therapeutic window, total treatment time and likely temperature rise characteristics for the RPE layer and adjacent Neuro-retina as an aid to optimize the selective damaging of the RPE layer while sparing cells and structures within the neuro-retinal and choroid.

FIG. 5 shows the cross-sectional structure of the human eye in the region of the RPE layer 50 and indicates the desired treatment zone 51 and surrounding zones 52.

Selective RPE treatment is dependant on the relative laser radiation absorption ratio between the neuro-retina and RPE layer and the physical characteristics of the layers, which can vary over a wide range from patient to patient. In addition, to achieve the selective coagulation of the thin, sub-surface RPE layer without causing collateral damage to the overlying neuro-retina requires a careful balance of interdependent treatment parameters. This makes it very difficult for the ophthalmic surgeon to choose the optimum treatment parameters and understand the combined effects. The interdependent parameters include treatment spot size, pulse width, pulse amplitude, pulse repetition rate and the total number of pulses delivered. All these parameters must be chosen to optimize the therapeutic window, and this must be judged against the total treatment time. If the treatment time which results from the parameters chosen to optimize the therapeutic window is too long the treatment effectiveness can be compromised by patient eye movement.

To allow the RPE layer of the retina to be selectively damaged by laser radiation, while sparing the overlying neuro-retina and underlying choroid, the laser treatment parameters must be carefully chosen. The relationship between these parameters and the resulting thermal effects within retinal layers is not easily understood, however it is possible to calculate these relationships in a way that can predict the likely clinical outcome and enable the impact of any changes to the treatment parameters to be assessed and presented to the ophthalmic surgeon in a meaningful and easily interpreted manner.

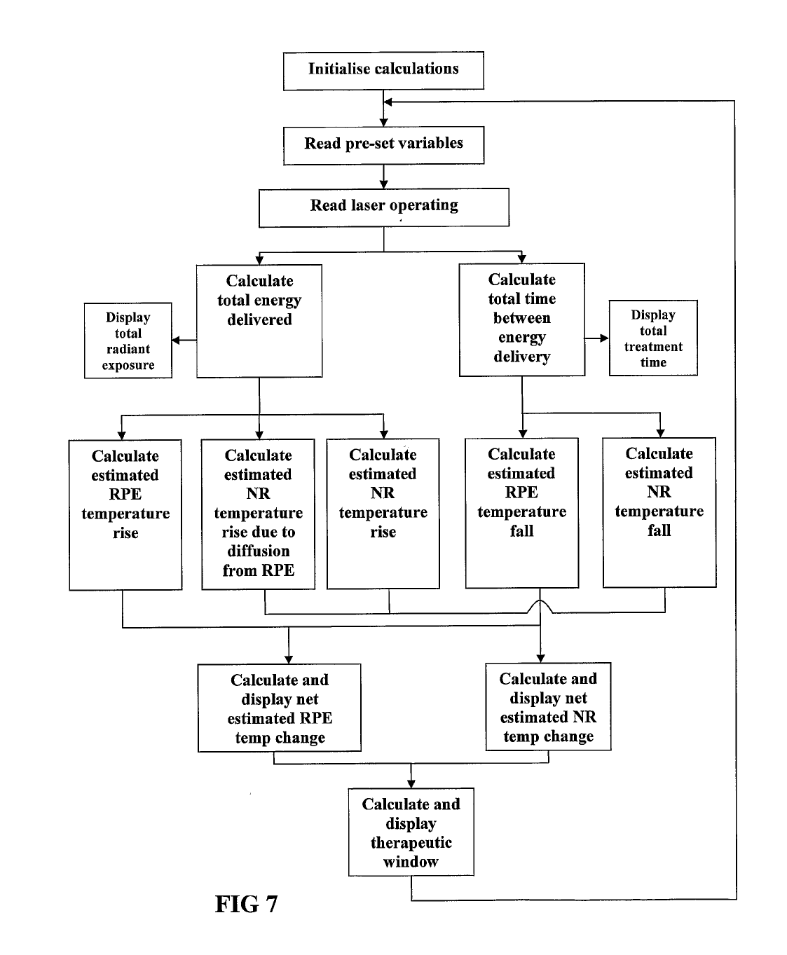

The aim of selective retinal treatment is to reach the cell rupture temperature within the RPE layer, by applying a series of laser pulses, while limiting the temperature in the neuro-retina immediately next to the RPE layer to the lowest possible value at the end of the laser treatment. The ratio between the RPE layer temperature and the neuro-retina temperature immediately next to the RPE layer is considered in this context to be the therapeutic window (TW). The greater the difference between the RPE temperature and the neuro-retina temperature the more selective the effect will be and therefore the wider the therapeutic window is considered to be. The thermal modelling principles are described in greater detail below with reference to the flowchart of FIG. 7.

Calculation of the therapeutic window is carried out as follows:

$$TW = \frac{{t}_{RPE} - ( {t}_{NR} \times {γ}_{RPE / NR} )}{{t}_{RPE}}$$

where

t_{RPE} is the cumulative temperature rise in the RPE melanin pigments caused by energy absorption during laser pulsing minus cumulative temperature drop between laser pulses due to diffusion;

t_{NR} is the cumulative temperature rise in the NR within the treatment zone at a point adjacent to the RPE layer caused by energy absorption during laser pulsing and heat diffusion from the RPE layer minus cumulative temperature drop between laser pulses due to diffusion; and

γ_{RPE/NR} is a pre-set scaling factor to account for the absorption ratio between the RPE and the NR. The scaling factor is chosen to give an approximately equal weighting to the RPE and NR temperatures.

The cumulative temperature rise in the RPE melanin pigments is dependant on:
Pulse duration (μs)
Pulse amplitude (W/pulse)
Total number of pulses (n)
Spot size (μm)
Relative absorption coefficient of the RPE.

The cumulative temperature drop in the RPE melanin pigments due to diffusion is dependant on:
Pulse repetition rate (ms)
Amplitude of temperature rise compared to ambient
Relative diffusion coefficient of the RPE.

The cumulative temperature rise in the NR is dependant on:
Pulse duration (μs)
Pulse amplitude (W/pulse)
Total number of pulses (n)
Spot size (μm)
Relative absorption coefficient of the NR
Heat diffusion from RPE.

The cumulative temperature drop in the NR due to diffusion is dependanton:
Pulse repetition rate (n)
Amplitude of temperature rise compared to ambient
Relative diffusion coefficient of the NR.

By displaying the relative TW based on the parameters selected by the user, the impact of any changes to the parameters can be quickly assessed. In addition, the likely effect over the course of the pulse train can be graphically displayed so that the relative changes in the RPE and NR temperatures, that result in the TW value, can be separately viewed.

Finding the optimum therapeutic window can involve changes to both the pulse repetition rate and the number of pulses delivered, which will affect the total treatment time. If the total treatment time is too long the treatment can be compromised by patient eye movement which can result in an insufficient treatment dose, particularly in the periphery of the treatment area. By determining and presenting the total treatment time to the user, along with the therapeutic window, the two factors can be assessed to give the best overall result.

The total treatment time can be calculated as follows:
Total treatment time=Total number of pulse bursts×Pulse burst repetition rate

By using this method the complex relationships between all laser parameters including, pulse duration, pulse amplitude, pulse repetition rate, total number of pulses and treatment spot size can be readily assessed by the ophthalmic surgeon in terms that directly relate to the objective of the treatment.

The parameters required for the calculation of likely temperature effects can be calculated from estimations of the thermal capacity and photoabsorption of the relevant tissues. The calculations are programmed into the system control processor in the form of an analysis algorithm within a package of treatment aid software which includes a graphical user interface, so that the likely treatment effect can be presented to the user to assist in the optimization of the treatment outcome. In order to assist understanding of the calculations required an example of a graphical user interface is shown in FIG. 6 and a flowchart of the thermal modeling used is shown in FIG. 7. As can be seen from FIG. 6, the temperature rise 61 in the RPE increases with each pulse until the RPE cell rupture temperature 62 is reached. The temperature rise in the neuro-retina 63 is much less with each pulse and remains below the damage threshold 64 for the neuro-retina. The selected pulse parameters 65 are adjusted to observe the effect on the thermal response of the RPE and the neuro-retina layers. The selected pulse parameters include the pulse width in μs, the pulse amplitude in Watts/pulse, the number of pulses per burst and the burst repetition rate in msec. The size 66 of the treatment zone is also entered. The aim is to achieve rupture of the RPE cells while avoiding damage to the neuro-retina layer. The calculated values are displayed in panel 67 and a graphical representation of the temperature rise is displayed in panel 68.

Pre-set treatment values are set in panel 69. The example in FIG. 6 has used the previously described visual lesion threshold (VLT) technique to determine an estimated RPE cell rupture temperature target. The VLT scaling factor is an empirical value based on personal patient factors such as ethnicity and age. It will be noted that the target RPE temperature rise 62 is 62.5 which is 313×0.2 (the VLT relative temperature rise times the VLT scaling factor). In this example the NR damage threshold 64 is derived from a fixed pre-set value due to relatively small patient to patient variations.

It will be appreciated that FIG. 6 is intended to be an interactive treatment aid which could be integrated as software into the control module or it could be operating within a separate computer which is a remote part of the control module via a conventional interface.

The interactive treatment aid software may include pre-programmed information on the normal range of parameter settings used for each treatment mode, derived from clinical trials, in order to display the treatment limits and advise the user if these are exceeded. For example, FIG. 6 is a display for Selective Retinal Therapy. The panel 70 displays treatment time and therapeutic window ranges that have been determined to be acceptable for this procedure. The calculated values are displayed on a bar graph so that the ophthalmic surgeon can easily see whether the selected laser treatment parameters will produce a desired result.

As mentioned above, the invention includes the ability to set and display treatment targets which may be derived from post treatment measurements of treatment effectiveness, internal estimated targets based on scaled visible treatment thresholds or external measurement systems of treatment effectiveness. For RPE treatment the targets would typically be in the form of a target minimum temperature rise value for the RPE layer to achieve cell damage, and a maximum target temperature rise value for the adjacent neuro-retina which should not be exceeded to avoid collateral damage. These target levels, shown in FIG. 6, allow actual treatment parameters to be chosen while the TW value is optimized to give the best margin for error for the treatment.

Other treatment modalities require similar displays that show other relevant parameters.

Once the user has selected the treatment parameters the user locks in the total radiant exposure value via the control module, so that changes to the treatment spot size, which may become necessary during treatment of different areas, cause an automatic adjustment of the pulse energy to maintain the selected total radiant exposure.

FIG. 7 describes the steps used in the thermal modeling algorithm to derive the predicted temperature effects and treatment outcomes in FIG. 6. The total energy delivery time is the total time that energy is being delivered to the target and results in thermal rise due to absorption, while the total time between energy delivery is the rest time between pulses and is dependant on diffusion characteristics. The algorithm uses the pre-set variables and estimated tissue absorption characteristics to calculated the RPE temperature rise during energy delivery and the estimated temperature drop during the rest period. The difference in these calculations is the estimated net temperature change in the RPE. The same calculations are made for the NR to obtain the estimated net temperature change in the NR, however in this case an additional allowance must be made for thermal diffusion from the RPE which is highly dependant on pulse duration. By comparing the predicted temperature rise in the target tissue (RPE) relative to the tissue which is to be protected (NR) a measure of the therapeutic window is derived.

From the above discussion it can be seen that the ophthalmic laser system is used in a method of carrying out a number of ophthalmic procedures such as Selective Retinal Therapy (SRT), Selective Laser Trabeculoplasty (SLT), Iridotomy and non-selective retinal coagulation. The method of treating the retinal pigmented epithelium layer of the retina of a patient using the SRT technique includes the steps of:
1. Selection, by the user, of the selective RPE treatment mode;
2. Selection of laser treatment parameters by the user;
3. Selection of patient dependant pre-set variables and treatment target values (if available);
4. Automatic calculation and display of the likely tissue effects and selectivity of the treatment to the RPE layer (the therapeutic window) which will result from the chosen parameters;
5. Automatic calculation and display of the total treatment time based on the chosen parameters;
6. Optionally adjusting the selected laser treatment parameters to achieve a desired selectivity and total treatment time;
7. Control and activation of the laser system, upon user command, to deliver the series of laser pulses to the treatment zone according to the selected laser treatment parameters.

Steps 4 and 5 are displayed using the graphical user interface of FIG. 6. The effect of step 6 is evident in the display in the graphical user interface.

As mentioned earlier, the method of treating the retinal pigmented epithelium layer of the retina of a patient can be expanded to use a visible lesion threshold to determine approximate selective RPE treatment parameters including the steps of:
1. Selection, by the user, of the selective RPE treatment mode;
2. Selection of laser treatment parameters by the user which are intended to cause a visible lesion at the periphery of the retina;
3. Selection of patient dependant pre-set variables including a Visual Lesion Threshold scaling factor;
4. Control and activation of the laser system, upon user command, to deliver the selected series of laser pulses to the periphery of the retina;
5. Adjustment of the treatment parameters by the user to determine the Visible Lesion Threshold (VLT);
6. Activation by the user of an automatic process which sets and displays the estimated optimal laser treatment parameters and tissue temperature rise targets for selective RPE treatment based on the VLT, VLT scaling factor and inbuilt parameter optimizing algorithm and displays the treatment target values;
7. Further manual adjustment of treatment parameters by the user if required;
8. Automatic calculation and display of the likely selectivity of the treatment to the RPE which will result from the chosen parameters;
9. Automatic calculation and display of the total treatment time based on the chosen parameters;
10.Control and activation of the laser system, upon user command, to deliver the selected series of laser pulses to the treatment zone.

The level of pigmentation in the RPE will directly influence the treatment parameters required to achieve selective RPE treatment. In human eyes the variation in average RPE pigmentation is about two fold and the method described in steps 4 to 8 above is designed to provide a means of compensating for this variation and providing the ophthalmic surgeon with estimated settings for effective selective RPE treatment that are chosen to suit each patient. By using the same duration laser pulses, but increasing the number of pulses per burst, a visible lesion can be produced in the periphery of the retina where no vision loss will occur. The total radiant exposure required to reach the threshold point where a visible lesion occurs will be approximately proportional to the individual level of RPE pigmentation in each patient so by applying a suitable scaling factor suitable selective RPE treatment parameters can be determined. An in-built parameter optimizing algorithm pre-sets the recommended treatment parameters, and then the user adjusts the setting manually if required. The algorithm will also display the calculated target temperatures for the RPE layer and adjacent neuro-retina so that the user can select other settings if required which will achieve the same selective temperature effects.

The value of the scaling factor can be determined by checking the treatment effectiveness using fluorescein angiography. A two fold variation in pigmentation also occurs between the fovea and paramacular regions with the fovea being the most heavily pigmented region. The scaling factor can also be adjusted to allow for this variation.

Another variation of the treatment method uses feedback from external measurement devices, which are designed to indicate the effectiveness of RPE selective treatment, to allow manual or automatic adjustment of treatment parameters to optimize the treatment including the steps of:
1. Selection, by the user, of the selective RPE treatment mode;
2. Connection of the laser system to a purpose built measurement system which can provide feedback on the effectiveness of selective RPE treatment;
3. Selection of patient dependant pre-set variables;
4. Adjustment of treatment parameters by the user;
5. Automatic calculation and display of the likely selectivity of the treatment to the RPE which will result from the chosen parameters;
6. Automatic calculation and display of the total treatment time based on the chosen parameters;
7. Control and activation of the laser system, upon user command, to deliver the selected series of laser pulses to the treatment zone;
8. Display of the treatment effectiveness based on the external measurement device and automatic or manual adjustment of treatment parameters to optimize the selective RPE treatment.

The invention is not limited to treatment of the retinal pigmented epithelium. Another application is treatment of the trabecular meshwork (TM) to lower the intra-ocular pressure using a procedure known as Selective Laser Trabeculoplasty (SLT), which is a treatment for open-angle glaucoma. Typical values are a wavelength of 532 nm, 1 μs pulse duration, 3 pulses per burst, 30 kHz pulse repetition rate, 50 μJ pulse energy, 1 kHz pulse burst repetition rate and a total of 50 bursts. Using a 200 micron diameter treatment spot this will produce a total radiant exposure of about 24 J/cm². The treatment would be repeated in about 50 spots around 180 degrees of the trabelular meshwork.

Melanin pigmented cells are contained within the trabecular meshwork which is directly accessible for laser treatment. The aim of the procedure is to selectively damage the pigmented cells while leaving the surrounding beams of the trabecular meshwork intact. While the overall method described for selective RPE treatment would be followed, the analysis algorithm, the information regarding normal treatment ranges and the method of determining treatment targets are adapted to suit selective trabecular meshwork treatment. By careful selection of the number of pulse bursts, the energy per pulse and the intervals between bursts, the selective damage to pigmented cells can be carried out in a far more controlled manner than with the delivery of a single high energy pulse.

Another treatment mode would be non-selective retinal coagulation which can be used to perform the well established retinal photo-coagulation tasks which often result in a visible lesion. Typical values are a wavelength of 532 nm, 1 μs pulse duration, 500 pulses per burst, 30 kHz pulse repetition rate, 50 μJ pulse energy, 60 Hz pulse burst repetition rate and a total of 3 bursts. This will produce a pseudo-CW mode which will deliver about 1.6W for 50 ms. When this mode of operation is selected the software will produce a simplified display which allows the user to select the output power and duration, with automatic conversion of the pulsing regime to suit.

Another treatment mode would be iridotomy which is a laser treatment for angle-closure glaucoma. The aim is to produce a hole in the iris to allow free flow of aqueous humor between the posterior and anterior chambers. This is a non-selective procedure with visible tissue effect so when this mode is selected the software will produce a simplified display showing normal treatment ranges and recommended pulse configurations.

The invention has been described primarily with reference to the particular embodiment of treating the retinal pigmented epithelium layer of the retina. It will be appreciated that other embodiments are envisaged within the spirit and scope of the invention.

1-34. (canceled)

35. An ophthalmic laser system comprising:
a laser module producing laser pulses with a pulse repetition rate capable of causing an additive thermal effect within target tissue, a pulse duration capable of containing thermal diffusion substantially within the target tissue, and a wavelength chosen to optimize energy delivery to the target tissue;
a control module in signal connection with the laser module and incorporating means for controlling said laser module to deliver a selected number of pulse bursts of selected duration and selected repetition rate with controlled pulse energy so that pulses within each burst have an additive thermal effect within the target tissue to cause an incremental temperature rise while limiting thermal diffusion to adjacent structures; and
a delivery module in optical connection with said laser module and signal connection with the control module, said delivery module delivering said bursts of laser pulses with a controlled radiant energy to a treatment zone.

36. The ophthalmic laser system of claim 35 wherein said laser module incorporates a pulsed laser and a pulse gating element said pulsed laser producing a train of pulses and said pulse gating element selecting bursts of pulses from said train of pulses.

37. The ophthalmic laser system of claim 36 wherein the pulsed laser operates at a pulse repetition rate from 1 kHz to 500 kHz, and a pulse duration from 0.1 μs to 40 μs.

38. The ophthalmic laser system of claim 36 wherein the pulsed laser operates in the wavelength range from 500 nm to 750 nm.

39. The ophthalmic laser system of claim 36 wherein the pulsed laser is a Q-switched solid state laser.

40. The ophthalmic laser system of claim 36 wherein the pulse gating element delivers a pulse burst repetition rate from 0.05 kHz to 5 kHz and from 1 to 100 pulses per burst.

41. The ophthalmic laser system of claim 36 wherein the pulse gating element delivers from 1 to 500 pulse bursts.

42. The ophthalmic laser system of claim 36 wherein the pulse gating element is an electro-optic switch.

43. The ophthalmic laser system of claim 36 wherein the pulse gating element is a fast switch of a power supply of the pulsed laser.

44. The ophthalmic laser system of claim 35 wherein the control module further comprises input means and display means.

45. The ophthalmic laser system of claim 35 wherein the delivery module comprises means for adjusting a spot size of said laser pulses.

46. The ophthalmic laser system of claim 36 further comprising feedback means that provides treatment feedback to the control module for dynamic control of the ophthalmic laser system.

47. An ophthalmic laser system comprising:
a laser module producing bursts of laser pulses with a pulse repetition rate from 1 kHz to 500 kHz, a pulse duration from 0.1 μs to 40 μs, a pulse burst repetition rate from 0.05 kHz to 5 kHz, and from 1 to 100 pulses per burst;
a control module in signal connection with the laser module and incorporating means for controlling said laser to deliver a selected number of laser pulses within a pulse burst of controlled pulse energy and a selected number of pulse bursts of controlled repetition rate; and
a delivery module in optical connection with said laser module and signal connection with the control module, said delivery module delivering said bursts of laser pulses with a controlled radiant energy to a treatment zone.

48. The ophthalmic laser system of claim 47 wherein the laser module produces from 1 to 500 pulse bursts.

49. The ophthalmic laser system of claim 47 wherein the laser module operates at a wavelength between 500 nm and 750 nm.

50. The ophthalmic laser system of claim 47 wherein said laser module incorporates a pulsed laser and a pulse gating element said pulsed laser producing a train of pulses and said pulse gating element selecting bursts of pulses from said train of pulses.

51. An ophthalmic laser system comprising:
a laser module producing bursts of laser pulses with a pulse repetition rate from 1 kHz to 500 kHz, a pulse duration from 0.1 μs to 40 μs, a pulse burst repetition rate from 0.05 kHz to 5 kHz, and from 1 to 100 pulses per burst;
a control module in signal connection with the laser module and incorporating processing means for predicting likely temperature effects and calculating a therapeutic window and total treatment time based on selected laser treatment parameters and allowing automatic or manual control of said laser module to deliver a selected number of laser pulses within a pulse burst of controlled pulse energy and a selected number of pulse bursts of controlled repetition rate in accordance with said therapeutic window and said total treatment time; and
a delivery module in optical connection with said laser module and signal connection with the control module, said delivery module delivering said bursts of laser pulses with a controlled radiant energy to a treatment zone.

52. A method of ophthalmic laser treatment including the steps of: selecting laser treatment parameters;
automatically calculating and displaying a likely selectivity and tissue temperature rise of a treatment which will result from the laser treatment parameters;
automatically calculating and displaying a total treatment time based on the laser treatment parameters;
adjusting said laser treatment parameters to achieve a desired selectivity, tissue temperature rise and total treatment time; and
controlling a laser system according to said laser treatment parameters to deliver laser pulses to a treatment zone.

53. The method of claim 52 further including the step of selecting target treatment values and displaying the target treatment values with the selectivity and likely tissue temperature rise.

54. The method of claim 53 wherein the step of selecting target treatment values includes selecting said target treatment values from a database of target treatment values obtained from one or more of: post treatment measurements of effectiveness; scaled visible treatment thresholds; or external measurement systems.

55. The method of claim 52 further including the step of determining target treatment values from patient dependant pre-set variables and measured values.

56. The method of claim 55 wherein the patient dependent pre-set variables are selected from one or more of: visual laser lesion threshold and visual lesion threshold scaling factor.

57. The method of claim 52 wherein the step of selecting laser treatment parameters includes the steps of:
selecting laser treatment parameters intended to cause a visible lesion at a periphery of a retina;
selecting patient dependant pre-set variables including a Visual Lesion Threshold scaling factor;
controlling and activating a laser system to deliver a selected series of laser pulses to the periphery of the retina;
adjusting the laser treatment parameters to determine the Visible Lesion Threshold; and
calculating and displaying the estimated optimal laser treatment parameters and tissue temperature rise targets for selective treatment based on the Visible Lesion Threshold and Visible Lesion Threshold scaling factor.

58. The method of claim 52 wherein the step of selecting laser treatment parameters includes selecting values for one or more of: laser pulse width; laser pulse amplitude; number of pulses per burst; total number of bursts; and pulse burst repetition rate.

59. The method of claim 58 wherein the number of pulses per burst is selected to be between 1 and 100.

60. The method of claim 58 wherein the number of pulse bursts is selected between 1 and 500.

61. The method of claim 58 wherein the pulse burst repetition is selected between 0.05 kHz and 5 kHz.

62. The method of claim 52 further the steps of:
connecting the laser system to an external measurement device providing feedback on the effectiveness of selective treatment;
displaying treatment effectiveness based on the external measurement device; and
adjusting treatment parameters to optimize the selective treatment.

63. A method of ophthalmic laser treatment of the retinal pigmented epithelium layer in a procedure such as Selective Retinal Therapy (SRT) including the steps of:
selecting laser treatment parameters;
automatically calculating and displaying a therapeutic window of a treatment which will result from the laser treatment parameters;
automatically calculating and displaying a total treatment time based on the laser treatment parameters;
adjusting said laser treatment parameters to achieve a desired tissue temperature rise and selectivity and total treatment time; and
controlling a laser system according to said laser treatment parameters to deliver laser pulses to a treatment zone.

64. The method of claim 63 wherein the therapeutic window is calculated from:
$$TW = \frac{{t}_{RPE} - ( {t}_{NR} \times {γ}_{RPE / NR} )}{{t}_{RPE}}$$
where
t_{RPE} is the cumulative temperature rise in the RPE melanin pigments caused by energy absorption during laser pulsing minus cumulative temperature drop between laser pulses due to diffusion; t_{NR} is the cumulative temperature rise in the NR within the treatment zone at a point adjacent to the RPE layer caused by energy absorption during laser pulsing and heat diffusion from the RPE layer minus cumulative temperature drop between laser pulses due to diffusion; and
γ_{RPE/NR} is a pre-set scaling factor to account for the absorption ratio between the RPE and the NR.

65. The method of claim 63 wherein the step of selecting laser treatment parameters includes the steps of:
selecting laser treatment parameters intended to cause a visible lesion at a periphery of a retina;
selecting patient dependant pre-set variables including a Visual Lesion Threshold scaling factor;
controlling and activating a laser system to deliver a selected series of laser pulses to the periphery of the retina;
adjusting the laser treatment parameters to determine the Visible Lesion Threshold; and
activation of an automatic process which calculates and displays the estimated optimal laser treatment parameters and tissue temperature rise targets for selective treatment based on the Visible Lesion Threshold and Visible Lesion Threshold scaling factor.

66. The method of claim 63 further including the steps of:
obtaining a measure of treatment effectiveness from at least one external measurement devices;
displaying treatment effectiveness based on the external measurement device; and
adjusting the laser treatment parameters to optimize the selective treatment.

67. A method of ophthalmic laser treatment of the trabecular meshwork in a procedure such as Selective Laser Trabeculoplasty (SLT) including the steps of:
selecting laser treatment parameters;
automatically calculating and displaying likely tissue effects and a therapeutic window of a treatment which will result from the laser treatment parameters;
automatically calculating and displaying a total treatment time based on the laser treatment parameters;
adjusting said laser treatment parameters to achieve a desired tissue temperature rise, selectivity, total treatment time and a total radiant exposure in the range from about 10 to 200 J/cm²; and
controlling a laser system according to said laser treatment parameters to deliver laser pulses to a treatment zone.

68. A method of ophthalmic laser treatment of the iris or retina in non-selective procedures such as Iridotomy or Pan Retinal Photo-coagulation (PRP) including the steps of:
selecting laser treatment parameters;
automatically calculating and displaying likely tissue effects which will result from the laser treatment parameters;
automatically calculating and displaying a total treatment time based on the laser treatment parameters;
adjusting said laser treatment parameters to achieve a desired tissue effects and total treatment time; and
controlling a laser system according to said laser treatment parameters to deliver laser pulses to a treatment zone.

69. The ophthalmic laser system of claim 35, the control module further incorporating a processing means for predicting likely temperature effects and calculating a therapeutic window.

70. The ophthalmic laser system of claim 47, the control module further incorporating a processing means for predicting likely temperature effects and calculating a therapeutic window
